(19) United States
(12) Patent Application Publication
(10) Pub. No.: US 2005/0060032 A1
(43) Pub. Date: Mar. 17, 2005

(54) Accommodating intraocular lens

(76) Inventors: Peter Magnante, Brookfield, MA (US); Mary Magnante, Brookfield, MA (US); David Miller, Brookline, MA (US); Ernesto Blanco, Belmont, MA (US)

Correspondence Address: ROBERT F. I. CONTE;BARNES & THORNBURG, P. O. BOX 2786, CHICAGO, IL, 60690, US

(21) Appl. No.: 10/738,271

(22) Filed: Dec. 17, 2003

Related U.S. Application Data
(63) Continuation-in-part of application No. PCT/US02/19534, filed on Jun. 21, 2002.
(60) Provisional application No. 60/299,757, filed on Jun. 22, 2001.

(51) Int. Cl. A61F 2/16

(52) U.S. Cl. 623/6.34; 623/6.37; 623/6.44

(57) ABSTRACT

An intra ocular lens arrangement having positive and negative lens elements which move during the eye's accommodation response in order to improve the image on the retina of objects viewed by the eye over a wide range of distances. The positive and negative lens elements either can be linked mechanically to constrain their relative movements or not linked. The lenses are positioned by an operating surgeon following cataract extraction in either the eye's ciliary sulcus or lens capsule. Alternatively, one of the lenses may be inserted into an eye that already has a lens implanted therein to further improve a person's vision. An improved intra ocular lens has is an optic lens having at least two pairs of haptics that controls the movement of the optic lens along the optical axis of the eye in response to the movement of the ciliary muscle of the eye acting on the haptics during the accommodation response, one pair of haptics having one end hinged to the lower half of the optic lens and the second end connected to an upper portion of the ciliary muscle, and a second pair of haptics hinged to an upper half of the optic lens and to a lower potion of the ciliary muscle.

This is a continuation-in-part of our PCT application PCT/US02/19534 filed Jun. 21, 2002 which claims priority of our US provisional application 60/299,757 filed Jun. 22, 2001.

### FIELD OF THE INVENTION

This invention relates to intra ocular lenses and more particularly to intra ocular lenses that have a positive and negative lens that may be assembled within the eye as part of implantation or outside of the eye.

### BACKGROUND

The lens within the human eye has the capability of changing shape and thereby focus so that objects both far and near can be registered sharply on the retina. This ability to change focus is known as accommodation. With age, the lens gradually loses its range of accommodation. The human lens not only loses accommodative range with aging, but also transparency. When the lens loses a significant amount of transparency (thus producing a blurry image on the retina), it is said that the lens is cataractous or has become a cataract. Treatment for a cataract requires the surgical removal of the cataract and the placing of a man made synthetic lens (intra ocular lens or IOL) in the eye. The earlier IOL's had a fixed focus and thus had no accommodative function.

However, in time a number of IOL's were designed in multifocal form. Different zones of a multifocal IOL have different dioptric powers. With such multifocal IOL's, light from objects, only within a specific range of viewing distances, passing through a particular zone will form sharply focused images on the retina. On the other hand, if an object is outside this range, its image formed by the zone under consideration will be blurry. Multifocal IOL's typically have two or more zones, each designed for a specific viewing distance. A consequence of this design approach is that the imagery of multifocal IOL's is never very sharp. The success of multifocal IOL's depends on the visual processing system of the patient's eye and brain that tends to pay attention to the light most sharply focused on the retina, and tends to ignore the light formed diffusely on the retina.

These were followed by IOL's that could move back and forth via ciliary muscle contraction and thus focus objects from different distances onto the retina. However, these IOL's have limited range of movement and thus a limited accommodative range.

Another form of IOL is made of an elastomer filled flexible balloon which is placed within the emptied lens capsule and alters lens shape under the influence of the ciliary muscle contraction.

Another accommodative IOL design is comprised of two positive lens elements (i.e. two plano-convex lenses) connected by two flexible hinges. The lens components are spread or come together in response to ciliary muscle contraction.

In our invention, we have an intra ocular lens that is a combination of a positive lens (i.e. lens is thicker at center than at edge), and a negative lens (i.e. lens is thinner at center than at edge). The positive-negative doublet combination of our invention yields a much larger focusing range with small changes in separation between the component lenses, when compared to either a positive singlet configuration or a positive-positive doublet configuration. Also, the newly designed IOL can alter dioptric power if placed in either of two intra ocular locations after cataract removal: a) within the capsular bag, or b) placed within the ciliary sulcus. In both locations, the contraction of the ciliary muscle alters the separation between the positive and negative lenses.

### SUMMARY OF INVENTION

The present invention provides:

1. Intra ocular lenses having the combination of a negative lens and a positive lens and forming a dual intra ocular lens in the eye by separately implanting the positive lens and the negative lens in the eye in such a manner that the positive lens will move relative to the negative lens along the optical axis in response to the movement of the ciliary muscle of the eye during accommodation response of the eye.

2. Intra ocular lenses having the combination of a negative lens and a positive lens which are joined together outside of the eye in such a manner that when the combination is implanted in the eye, the positive lens will move relative to the negative lens another along the optical axis in response to the movement of the ciliary muscle of the eye during accommodation response of the eye.

3. Intra ocular lenses having the combination of a negative lens and a positive lens and forming a dual intra ocular lens in the eye by implanting a positive lens or a negative lens into an eye already having implanted therein one of the lenses.

4. An intraocular lens having a lens linkage that provides relatively larger movement of the lens with a small movement of the ciliary muscle.

5. An intraocular lens having a first linkage having a first end connected to the lower portion of the intraocular lens and a second end to be connected to an upper eye portion of the ciliary muscle, and a second linkage having its first end connected to the upper portion of the intraocular lens and its second end to be connected to a lower eye portion of the ciliary muscle.

6. Intra ocular lenses as noted in above 1- 5 wherein the lenses are implanted in or outside of the lens capsule or capsular bag.

One embodiment of the present invention is to provide dual intra ocular lenses having the combination of a negative lens and a positive lens substantially coaxially aligned and separated along their optical axis and forming the dual intra ocular lens in the eye by separately implanting the positive lens and the negative lens in the eye such that the positive lens will move relative to the negative lens.

A second embodiment of the present invention is to provide an eye intra ocular lens that has a negative lens and a positive lens that are axially separated and said intra ocular lens is formed inside the eye as part of an implantation of the negative and positive lenses in an eye or outside of the eye by connecting the negative and positive lenses prior to implantation into the eye such that the positive lens will move relative to the negative lens.

A still further embodiment of the present invention is to provide a method of improving vision for an eye which has been diagnosed as being approved for intra ocular lens implants comprising implanting a negative lens with, before or after implanting a positive lens, and implanting said negative lens such that the positive lens will move relative to negative lens along the optical axis in response to the ciliary muscle of the eye during the accommodation response of the eye.

For the purpose of promoting an understanding of the principles of the invention, references will be made to the embodiment illustrated in the drawings. Specific language will also be used to describe the same. It will, nevertheless, be understood that no limitation of the scope of the invention is thereby intended, such alterations and further modifications in the illustrated device, and such further applications of the principles of the invention illustrated herein being contemplated as would normally occur to one skilled in the art to which the invention relates.

### DESCRIPTION OF DRAWINGS

FIG. 1 illustrates the two-lens system design (front element a positive lens, rear element a negative lens). The lenses are significantly separated so as to focus the image of a relatively nearby object onto the retina.

FIG. 1A illustrates the two-lens system design (front element a negative lens, rear element a positive lens). The lenses are significantly separated so as to focus the image of a relatively nearby object onto the retina.

In FIG. 2, the lens elements are shown closer together as a result of the relaxation of the ciliary muscle, allowing for the sharp focus of images of relatively distant objects onto the retina.

FIG. 3 shows one possible configuration when the lens elements are mechanically linked by a hinged haptic which causes the two lenses to separate.

In FIG. 4, the focal length of the system can be changed by changing the separation of the lens elements.

In FIG. 5, the method in which the ciliary muscle couples to the hinged haptic is shown when both lens components of the IOL are placed in the ciliary sulcus.

In FIG. 6, both lens components of the IOL are placed within the capsular bag where both the constriction of the ciliary muscle and the elasticity of the lens capsule provide the forces which determine the separation of the two lenses.

FIG. 6A shows the connection of the IOL to the ciliary sulcus with the IOL hinged haptics of the present invention.

FIG. 6B shows the connection of the IOL placed within the capsular bag to the ciliary sulcus with the IOL hinged haptics of the present invention.

FIG. 6C shows the IOL haupic levers of the present invention as connected to the ciliary sulcus.

FIG. 6D shows the IOL haupic levers of the present invention as connected to the IOL.

FIG. 7 shows an optical ray trace of a positive singlet lens located to focus sharply on the retina an image of an object located an infinite distance away.

FIG. 8 shows an optical ray trace of the same singlet lens of FIG. 7 shifted 1.92 mm to the left for 3 diopters of accommodation.

FIG. 9 shows an optical ray trace of a positive-negative doublet lens in contact which forms a sharply focused image on the retina of an object at infinity.

FIG. 10 shows an optical ray trace of the same doublet lens of FIG. 9 separated by 0.87 mm for 3 diopters of accommodation.

FIG. 11 shows an optical ray trace of a pair of equal positive lenses in contact which forms a sharply focused image on the retina of an object at infinity.

FIG. 12 shows an optical ray trace of the same positive-positive doublet of FIG. 11 separated by 1.75 mm for 1.25 diopters of accommodation.

### DETAILED DESCRIPTION OF INVENTION

Our invention relates to an IOL configuration having a positive lens and a negative lens with a variable focal length (or dioptric power) that depends on the distance along the optical axis separating the two lenses while maintaining a constant angular magnification for objects viewed over a wide range of distances (e.g. from infinity to typical reading distances). The positional order of the lenses in the eye can be either with the positive lens more anterior or the reverse, or with the negative lens more anterior or the reverse. Each negative and positive lens may be placed either in the capsular bag or the ciliary sulcus. The negative and positive lenses either may or may not be mechanically linked to one another by tabs and strut-like linkages (haptics) attached to the edges of the two lenses. During cataract surgery and IOL implantation, the positive and negative lenses may be inserted intra ocularly either one at a time (if the components are not mechanically linked to one another), or both at the same time (if the components are mechanically linked to one another). The linkages serve to hold the positive and negative lenses in place, as well as serve to adjust and control the distance separating the two lenses when powered by ciliary muscle contraction. It is the separation between the lenses that accounts for the change in IOL power (i.e. accommodation).

The lenses are located with their axes parallel (or nearly parallel) to one another and to the optical axis of the eye (coaxial configuration). This coaxial configuration is maintained during the change in separation of the lens elements which causes the eye's accommodative response. The positive-negative lens configuration provides a greater change of dioptric power with change in separation distance than any other configuration such as a positive-positive or a singlet positive configuration.

However, we also provide a linkage for our intraocular lens that provides larger axial movement of the lens than known linkages for IOLs. Our linkage can be used on either the negative or positive lens when a dual lens is used or on a single lens.

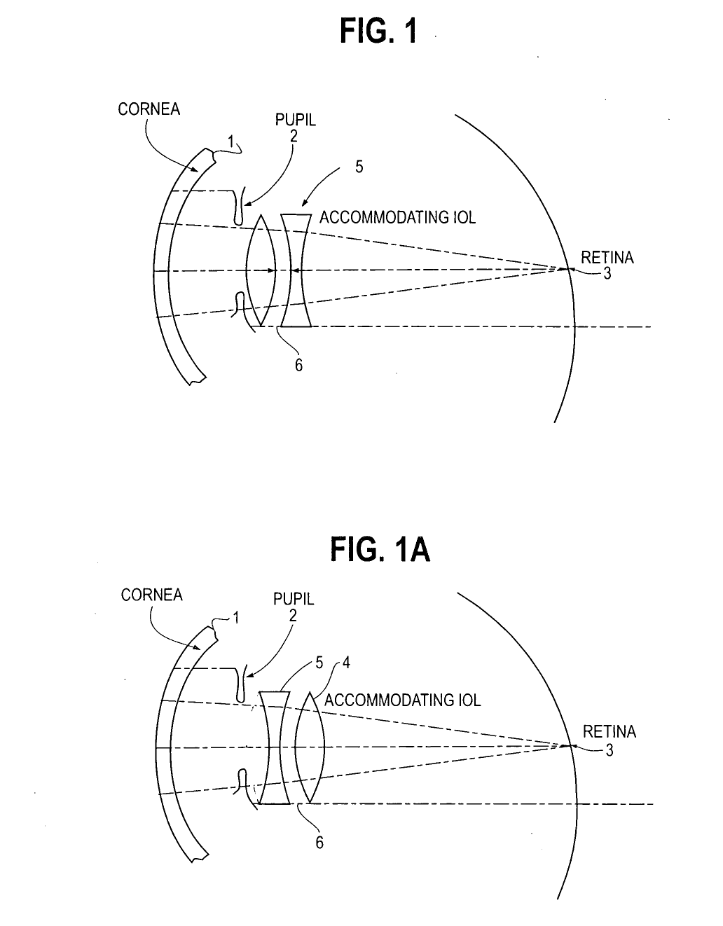

One general configuration of our dual intra ocular lens within the eye is shown in FIG. 1 when the eye is focused on a nearby object. The eye is represented schematically by the cornea 1, the pupil 2, and the retina 3. The dual IOL's optical components, are a positive lens 4, and a negative lens 5, that are situated just behind pupil 2, with the negative lens 5 more anterior. In this position, the ciliary muscle is somewhat contracted separating the negative lens 5 away from positive lens 4 to provide a space 6.

FIG. 1A illustrates another general configuration of the dual IOL within the eye. In this configuration, the positive lens 4 is more anterior. The ciliary muscle is somewhat contracted and moves the positive lens 4 away from the negative lens 5 to provide a space 6.

The positive and negative lenses 4,5 generally will have spherical surfaces; however, since astigmatic and other aspherical-shaped singlet IOL's (both symmetric and asymmetric with respect to their optical axes) now are manufactured for implantation in the eye, the positive and negative lenses 4,5 may also have these more general surface shapes. Fresnel-type IOL lenses also are used in cataract surgery. These lenses generally have a succession of stepped-annular zones or facets which serve to minimize a Fresnel lens's thickness while maximizing it power. Fresnel-type positive and negative lenses are suitable lens components for use in our invention. Also, diffractive lens configurations are sometimes used (i.e., diffractive lenses or lenses with one surface diffractive and the other surface refractive.

Generally, a person is not reading and is looking at objects more than two feet away. In that condition, the ciliary muscle is relaxed and the general configuration of our dual IOL within the eye is shown in FIG. 2—the eye is focused on a distant object. The positive lens 10 and negative lens 11 are brought together with a slight space 12 there between. The spacing 12 is much less than the spacing 6 in FIG. 1. However, the spacing 12 is necessary to prevent the two lenses from adhering to each other. The reason why the IOL spacing 6 is larger when the eye's focus changes from viewing a distant object (FIG. 2) to viewing a nearby object (FIG. 1) may be understood by examining the well-known formula (Equa. 1) for the combined focal length of a pair of thin lenses, f, expressed in terms of the focal lengths of the two lens components, f1 and f2, and the spacing between them, d.
1/f=1/f1+1/f2−d/(f1 *f2) (1)
Let f1 and f2 represent the respective focal lengths of the positive and negative lens components. Since f1>0 and f2<0, Equa. 1 shows that f decreases as d increases. As the eye accommodates as shown in FIG. 1, its focal length needs to decrease (i.e. greater optical power) which corresponds to a larger spacing 6 than the spacing 12 needed for the unaccommodated eye shown in FIG. 2.

The easiest way to understand why a positive-negative doublet provides a greater change of dioptric power with change of separation distance than a positive-positive doublet is by examining the formula for the combined power of a pair of thin lenses, D, expressed in terms of the powers of the two lens components, D1 and D2, the spacing between them, d, and the refractive index of the medium, n, in which the lenses are situated. Multiply both sides of Equa. 1 by the refractive index, n, and then recognize that dioptric power is n/(focal length) in order to find Equa. 2.
D=D₁+D₂−D₁*D₂*d/n (2)

The change of dioptric power with change of separation distance, expressed as δD/δd, is obtained by differentiating Equa. 2.
δD/δd=−(D₁*D₂)/n (3)

When fitting a particular patient with an IOL, the doctor determines the correct IOL power for distance vision which, in terms of the above parameters, requires D₁+D₂ to have a particular value. By way of example, we will set D₁+D₂=24 diopters which is a typical value. Table 1 below shows δD/δd calculated from Equa. (3) for different values of D₁ and D₂ (constrained so that their sum equals 24 diopters) when the refractive index of the media n=1.33. Note in Table 1 that the largest values of δD/δd (i.e. the change of dioptric power with change of separation distance) occur when D₁ is most positive and D₂ is most negative.

TABLE 1

| | | | | | | | |
|---|---|---|---|---|---|---|---|
| D₁ (diopter) | 6 | 12 | 18 | 24 | 30 | 36 | 42 |
| D₂ (diopter) | 18 | 12 | 6 | 0 | −6 | −12 | −18 |
| δD/δd | | | | | | | |
| (diopter/m) | −81.2 | −108.3 | −81.2 | 0 | +135.3 | +324.8 | +568.4 |

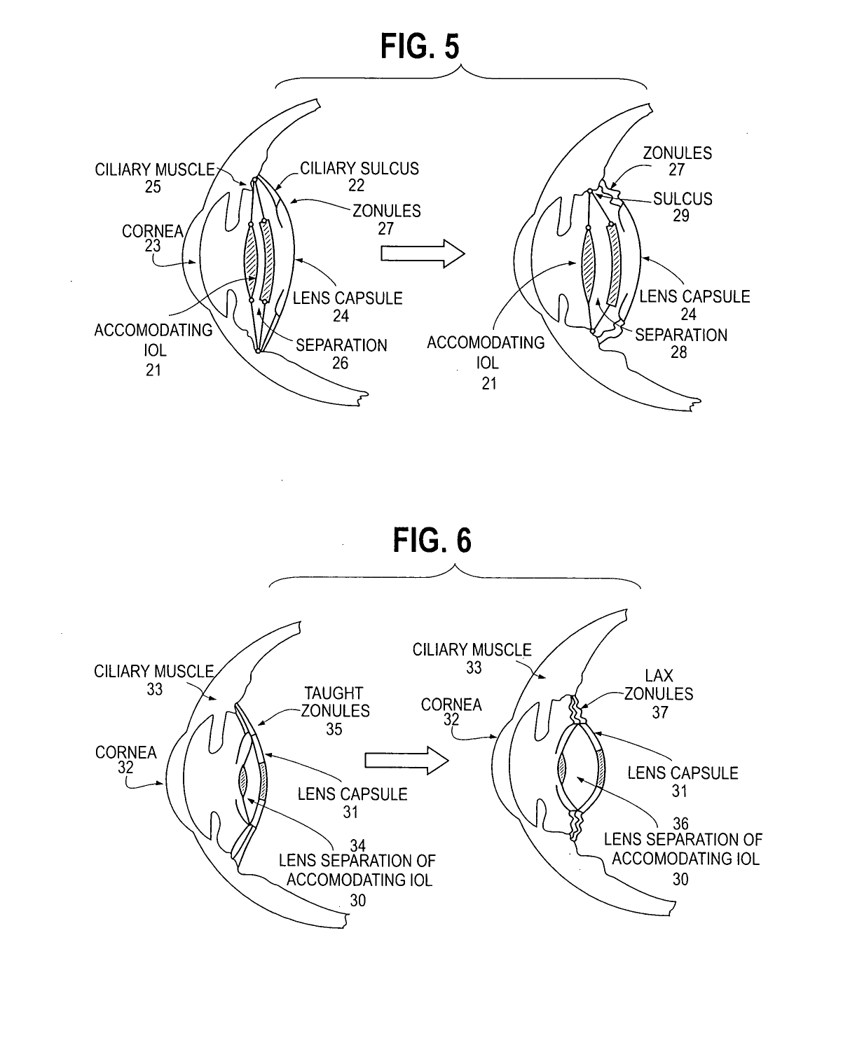

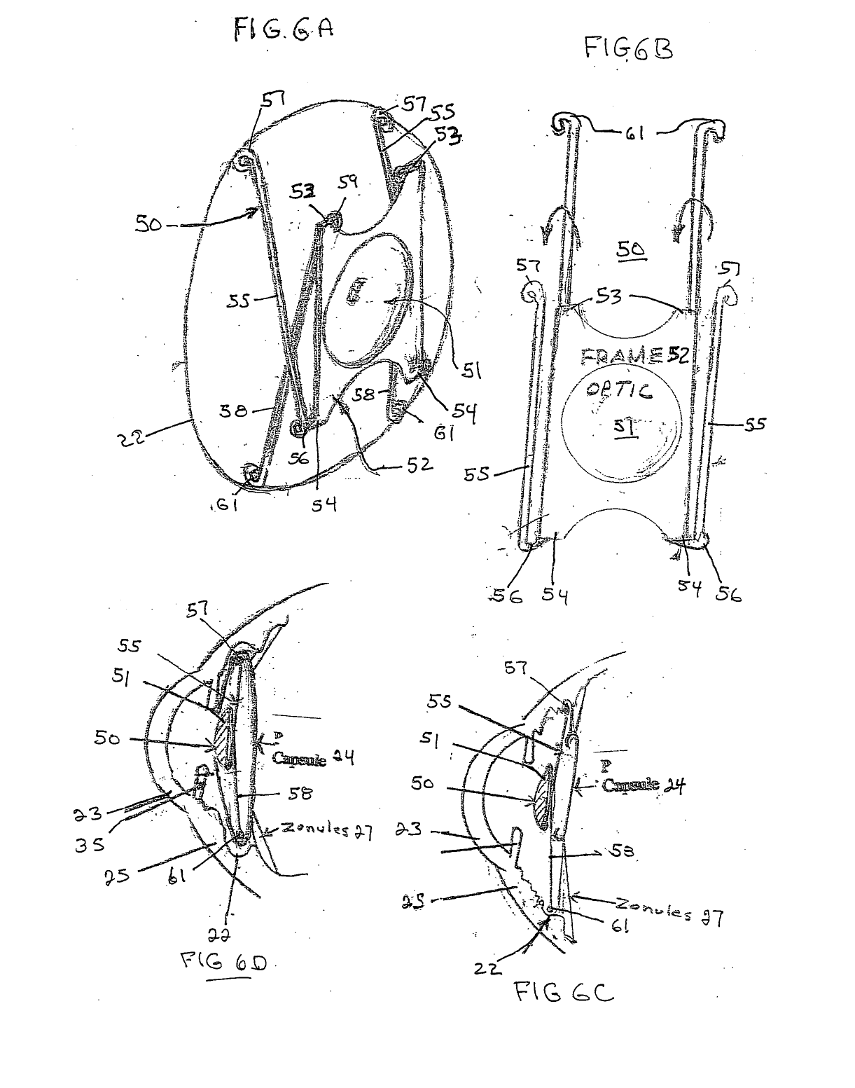

As noted above, the preferred manner of correcting a patient's vision in one eye is to open the eye's lens capsule or capsule bag 31 (FIG. 6), remove the eye lens and first insert the desired positive or negative lens in the lens capsule or capsule bag . Then the other lens is inserted into the lens capsule or capsule bag. The positive lens and negative lenses are connected to each other such that when the ciliary muscle contracts, the two lenses axially separate from each other and when the ciliary muscle relaxes, the two lenses axially move towards each other. In our invention generally, only one of the lenses ( preferably the positive lens) moves and the other lens ( the negative lens) does not move or moves substantially less and both lenses remain substantially coaxial with each other. One manner of connecting the two lenses to each other would be to connect them both independently to the ciliary muscle and the ciliary muscle zonules. Another method would be to attach the linkages of the positive lens to the linkages of the negative lens. The attachment could be any suitable attachment that would allow one IOL to move away from the each other IOL when the ciliary muscle contracts and towards the other IOL when the ciliary muscle relaxes.

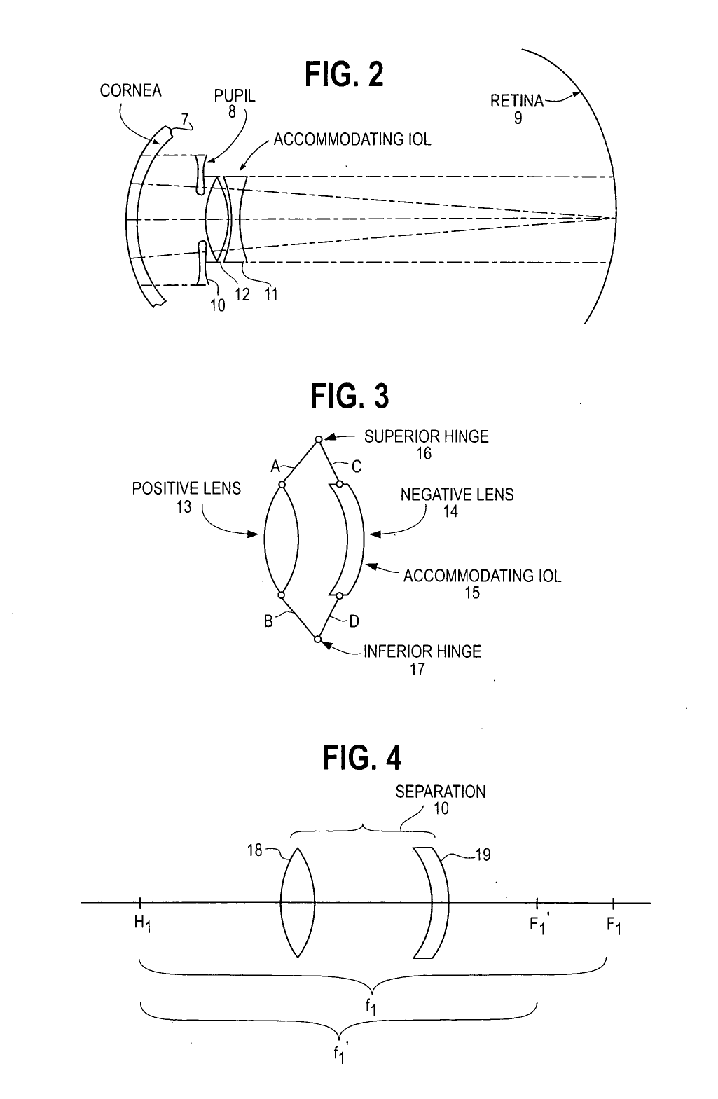

The linkages A, B, C, and D(FIG. 3) are sized to provide adequate leverage to cause the positive lens 13 and the negative lens 14 to separate when the ciliary muscle contracts. The linkages are generally made of the same material as their respective lens and are preferably integral with their respective lenses. They, of course, may be made of separate materials and appropriately affixed to their respective lenses. The linkages are sufficiently rigid such that a force directed towards the center of the eye by a contracting ciliary muscle causes the lenses 4,5 and 13,14 to separate from each other as shown in FIGS. 1, 1A, and 3.

FIG. 3 shows one possible configuration of a way in which a positive lens 13 may be coupled mechanically to a negative lens 14, where both lenses comprise an assembled accommodating dual IOL 15. The coupling may be accomplished by linkages A, B, C, D, made from the same polymer material from which their respective lenses are made. The linkages also can be made from other materials as noted above. In FIG. 3, two hinges are shown, a superior hinge 16 and an inferior hinge 17; however, more than two hinges may be used to achieve the intended movement of the positive and negative lenses. As shown in FIG. 3, each hinge consists of a pair of semi-rigid straight (or reasonably straight) linking arms and three flexure joints (one at the apex of the pair of linking arms A, B, C, D, and one each where a linking arm is attached to a lens). The configuration shown in FIG. 3 will cause the lenses to separate when a compressive force is applied between the two hinges.

In FIG. 3 the linking arms are appropriately joined at their apexes. However, although the joining of the linkages is preferred, the positive lens linkages A, B, and the negative lens linkages C, D may be separate and not attached. However, they will extend at an angle to the optical axis so that at least one of the lenses can move along the optical axis .

Although the hinge configuration in FIG. 3 shows that the linking arms have approximately the same length and that each link is angled so that a pair forms a “V” (or “inverted-V” shape) at its apex, linking arms having different lengths and different angles from those shown in FIG. 3 also may be used to achieve the purposes of the invention.

Another hinge configuration that may be used to move the two lenses during accommodation can have a more general “lambda” shape (i.e. the Greek letter λ) or, perhaps, a mirror-image λ shape. This kind of hinge has four (not three) flexure joints and, with a generalized λ-hinge configuration, the legs may have different lengths and angles. Within the practice of mechanical engineering and design, it is obvious to those skill in those fields that there are many other hinge configurations that will result in constraining the movements of the two lenses appropriately in order to achieve the benefits of our invention.

Although FIG. 3 shows the positive and negative lens components of the IOL coupled by mechanical linking arms, two independent (i.e. not linked) lenses conceivably can be implanted in sequence by skilled surgeons at precise locations in either the capsular bag or the ciliary sulcus to achieve good focusing during accommodation.

FIG. 4 illustrates the change of the focal point when the positive lens 18 and the negative lens 19, initially in close proximity, are moved apart to a prescribed separation 20. Initially the negative lens 19 is to the left of its location shown in FIG. 4 and similar to the position shown in FIG. 2 wherein the negative lens is almost in contact with positive lens 18. In this initial configuration, the focal point is at F1 and the focal length with respect to the principal plane at H1 is f1. When the lenses have separation 20 as shown in FIG. 4, the focal point is at F1′ and the focal length with respect to the principal plane at H1 is f1′. Note that with increased separation of the positive-negative doublet, the focal length decreases (i.e. dioptric power increases) in accord with Equation 1 and the discussion thereof.

Although the preferred two lenses are inserted into the eye separately, the two lenses could be joined prior to insertion to form a dual IOL and the dual IOL is inserted. This is not preferred because this requires a larger incision to be made after the cataract is removed.

FIG. 5 (left) shows an accommodating dual IOL 21, which is a mechanically linked positive-negative lens pair, implanted in the ciliary sulcus 22 behind the eye's cornea 23 and in front of the lens capsule 24 with the ciliary muscle 25 relaxed (eye focused at distant object). The dual IOL 21 is mechanically linked after or before being implanted. In this instance lens separation 26 is relatively small. The zonules 27 support the lens capsule 21 from which the cataract has been removed.

FIG. 5 (right) shows the same accommodating dual IOL 21 and how the lens separation 28 increases during accommodation when the ciliary muscle tightens causing the sulcus 22 to constrict. Also shown is how the lens capsule 24 and the supporting zonules 27 tend to move to the right during ciliary muscle contraction.

FIG. 6 (left) shows an accommodating dual IOL 30, which is a mechanically linked positive-negative lens pair, implanted in the lens capsule 31 behind the eye's cornea 32 with the ciliary muscle 33 relaxed (eye focused at distant object). As with IOL 21, IOL 30 is mechanically linked after or before implantation. In this instance, lens separation 34 is relatively small, since the zonules 35 which are taught exert an outward tension at the edges of the lens capsule 31 where the dual IOL's flexible hinged apex is attached.

FIG. 6 (right) shows the same accommodating IOL 30 implanted in the lens capsule 31 behind the eye's cornea 32, and how the lens separation 36 increases during accommodation when the ciliary muscle 33 tightens causing lax zonules 35 which exert reduced tension at the edges of lens capsule 31 where the IOL's flexible hinged apex is attached.

FIG. 6A and FIG. 6B show our IOL 50, having an IOL optic (lens) 51 attached generally in the center of an IOL frame 52. The frame 52 has two upper (superior) comers 53 and two lower (inferior) comers 54. A pair of superior haptics 55 (superior linkage legs) have their respective first ends 56 pivotally linked to their respective lower frame comers 54. The second ends 57 of the superior haptics 55 are attached to the upper or superior portion of the ciliary sulcus 22.

A pair of inferior haptics (inferior linkage legs) 58 have their respective first ends 59 pivotally linked to the upper frame comers 53. The respective second ends 61 of the inferior haptics 58 are attached to the lower or inferior portion of the ciliary sulcus 22. The haptics 55 as shown are generally straight and are a parallel extending pair as are the haptics 58. Although a separate frame 52 is used to hold the IOL optic 51, the frame could be an integral part of the IOL optic 51 or the haptics could be connected directly to the IOL optic 51. That is the haptics 55 would be connected to the lower portion of the IOL optic below the center line dividing the upper and lower portion of the IOL i.e., the center line passing through the 3 and 9 o'clock position. The first end 56 of the haptics 55 is preferably connected at a position from 8 to 6 o'clock and the first end 59 of haptic 58 is preferably connected at a position from 3 to 6 o'clock. The haptics 55 and 58 are connected too the IOL in such a manner that the IOL moves in the optical axial direction.

Although we show the use of two pairs of haptics, the use of only two haptics would be possible as long as they are connected to provide the axial movement. However, at least two pairs are preferable to insure axial movement. The closer the haptics are to the 6 and 12 o'clock positions, the greater the length of the haptics can be made if the haptics are placed on a diagonal.

FIG. 6C shows our IOL 50 implanted in the ciliary sulcus 22 behind the eye's cornea 23 and in front of the lens capsule 24. If a negative lens is also to be used, the negative lens can be either implanted separately into the eye or in combination with the IOL 50. The zonules 27 support the lens capsule 21 from which the cataract has been removed.

FIG. 6D shows the IOL 50, implanted in the lens capsule 24 behind the eye's cornea 27 and iris 35 and connected to the ciliary muscle 25. The zonules 27 either exert an outward or reduced tension at the edges of the lens capsule 24 where the IOL's haptic ends are connected.

Ray Traces for Accommodating IOL Models:

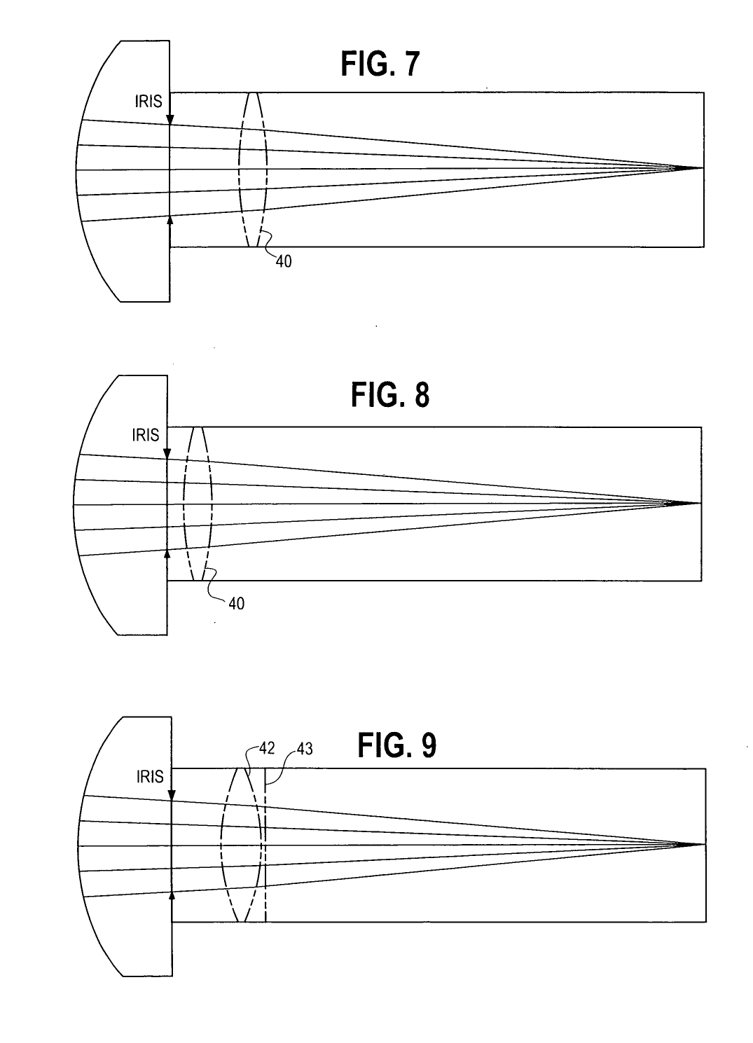

The following FIGS. 7-12 are ray traces from a computerized lens design program (ZEMAX) which illustrate the movement required from different types of accommodating IOL models for a prescribed amount of accommodation. All of the Figures use an eye having a cornea with a 8.00 mm radius of curvature. The iris has a 3.50 mm diameter and is located 3.60 mm from the cornea. The cornea to retina distance is 23.90 mm and except for the IOL, the media of the eye is water ( n=1.333).

FIG. 7 shows a positive single lens 40, (+24.1 diopter) located to focus sharply on the retina an image of an object located in air an infinite distance away from the cornea. The lens is made of PMMA ( n=1.492) and the lens posterior is 16.7 mm from the retina. The lens has a 1.0 mm center thickness.

FIG. 8 uses the same single lens 40, of FIG. 7 except shifts the lens 1.92 mm to the left (the posterior of the lens is 18.62 mm from the retina ) and the object in air is ⅓ m from the cornea for 3 diopters of accommodation (i.e. 0.64 mm/diopter).

FIG. 9 illustrates the calculation for a sharply focused image on the retina of an object at infinity for a positive-negative doublet with the posterior surface of the positive lens 42, being 16.7 mm from the retina and the object in air is an infinite distance from the cornea. The positive lens 42, has a +44 diopter power and a 1.5 mm center thickness, and the negative lens 43, has a −22 diopter power and a 0.2mm center thickness). The spacing between the lenses is 0.0 mm indicating that the two lenses are in contact which results in a sharply focused image on the retina of an object at infinity.

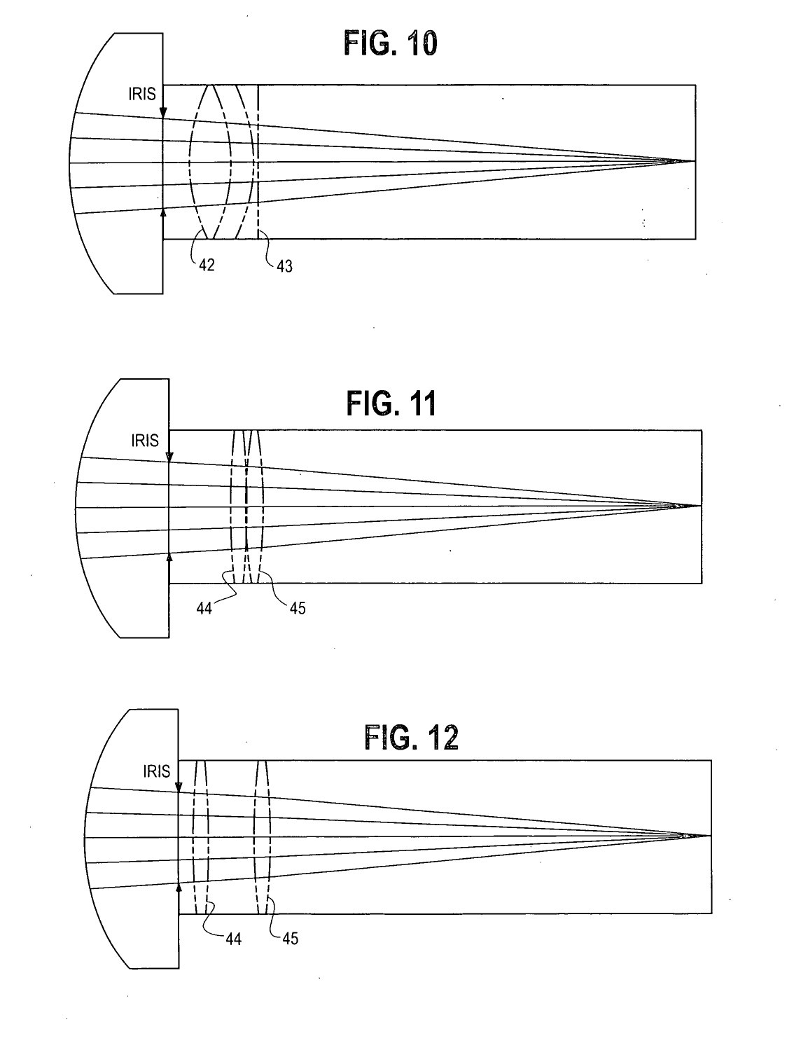

FIG. 10 illustrates the calculation for the same doublet lens of FIG. 9 with the posterior surface of the positive lens 42, being 16.7 mm from the retina and the object in air being ⅓ m from the cornea. The lenses are separated by 0.87 mm for 3 diopters of accommodation (i.e. 0.29 mm/diopter).

FIG. 11 illustrates the calculation for a sharply focused image on the retina of an object at infinity for a positive-positive doublet IOL with the posterior surface of the doublet being 16.7 mm from the retina and the object in air at an infinite distance from the cornea. Each of the equal positive lenses 44, 45, has +12 diopter power and a 0.6 mm center thickness. The spacing between the lenses is 0.0 mm indicating that the two lenses are in contact which results in a sharply focused image on the retina of an object at infinity.

FIG. 12 shows the same positive-positive doublet of FIG. 11 except the spacing between lenses is 1.75 mm for 1.25 diopters of accommodation (i.e. 1.40 mm/diopter).

By comparing the collective results for FIG. 9 and FIG. 10 (positive-negative doublet) with the collective results for FIG. 7 and FIG. 8 (positive single lens) and with the collective results for FIG. 11 and FIG. 12 (positive-positive doublet), note that the positive-negative doublet configuration provides a significantly greater change of diopter power with change in separation than does either of the other configurations.

Mathematical model results for Separation of Accommodating IOL Doublet Lens:

By applying the well-known lens formula (i.e. the equation that relates object and image distances to the focal length of a “thin” lens, namely
1/u+1/v=1/f)
successively to the eye's corneal surface, then to its anterior positive IOL component lens, and finally to its posterior negative IOL component lens, one can derive by algebraic manipulations the mathematical equation which gives the separation of the IOL component lenses in terms of the physical dimensions and optical characteristics of the eye's components as well as its accommodative state. The results of that derivation are presented here. Furthermore, the equation is applied to a specific model eye for several different powers for the positive and negative IOL components (i.e. D₁ and D₂).
The specific model eye is described as follows:
1) length from corneal apex to retina is .0.0239 meter,
2) positive IOL lens has power D₁ diopters and is more anterior Y i.e. closer to the cornea,
3) L₁ is the fixed distance from the cornea to the negative IOL lens (L₁=0.0072 meter),
4) negative IOL lens has power D₂ diopters and is a fixed distance L₂ from the retina (L₂=0.0167 meter),
5) corneal power D₀ is 41.625 diopter, and
6) refractive index, n, inside the eye is 1.333.

The accommodation power of the eye is the variable D′ and typically ranges from 0 to 3 diopters.

Next in Equation 4, we define the following parameters that have no special significance except to make the final equation, which is Equation 5, relatively compact. The spacing between the positive and negative component lenses, d, may now be written in terms of the known input and other defined parameters as Equation 5.
Define D*=D₀−D′ and A=(D₂/n−1/L₂)^{31 1}−L₁ (4)
d=L₁+½(A−n/D*)[1−{1+[4n (n/(D₁D*)+A(1/D*+1/D₁)]/(A−n/D*)²}^{1/2} (5)

Equations 4 and Equa. 5 were used to find the change in separation distance of the IOL component lenses per change in the eye's accommodative power, 5d/5D′, for several sets of D₁ and D₂ values. These results are expressed in Table 2.

TABLE 2

| | | | | | |
|---|---|---|---|---|---|
| D₁ | 43.8 | 30.0 | 30.0 | 25.0 | 25.0 |
| (diopter) | | | | | |
| D₂ | −22.2 | −10.0 | −5.0 | −10.0 | −5.0 |
| (diopter) | | | | | |
| δd/δD | 0.318 | 0.488 | 0.519 | 0.560 | 0.595 |
| (mm/diopter) | | | | | |

Note that the result given in the first row of Table 2 (i.e. 0.318 mm/diopter) is in fairly good agreement with the ray trace result given for a similar model eye (i.e. 0.29 mm/diopter) where D₁=+44 diopter and D₂=−22 diopter (see FIG. 9 and FIG. 10). The small difference is due to the fact that the mathematical model used in this section treats the lenses as “thin” whereas the ray trace results modeled finite thickness lenses. Furthermore, the results in Table 2 show that a positive-negative lens configuration tends to produce a larger accommodation change with lens displacement as the negative lens is made stronger.

Various features of the invention have been particularly shown and described in connection with the illustrated embodiment of the invention, however, it must be understood that these particular arrangements merely illustrate, and that the invention is to be given its fullest interpretation within the terms of the appended claims.

1. An eye intra ocular lens system comprising:
at least a positive lens and a negative lens that cooperate with each other to provide a corrected vision;
said negative lens is to be implanted with, before, or after the implantation of said positive lens;
said positive lens has positive lens means to move said positive lens relative to said negative lens in response to movement of the ciliary muscle of the eye during accommodation response of the eye; and wherein said movements during the accommodation response are along the optical axis of the eye and are controlled in order to improve the image on the retina of objects viewed by the eye over a wide range of distances.

2. The eye intra ocular lens system of claim 1 wherein said intra ocular lens system as implanted has a focal length that decreases as viewed objects move closer to the eye, and increases as viewed objects move farther from the eye.

3. The eye intra ocular lens system of claim 1 wherein the positive lens is to be located either in the eye's ciliary sulcus or lens capsule and the negative lens is located either in the eye's ciliary sulcus or lens capsule.

4. The eye intra ocular lens system of claim 3 wherein the positive and negative lenses can have any of the following types of surface shapes: spherical, astigmatic toric, aspherical with or without axial symmetry, multi-zoned surfaces as those found on Fresnel lenses, diffractive surfaces, and one surface diffractive and the other surface diffractive.

5. The eye intra ocular lens system of claim 4 wherein said negative and positive lens means are semi-rigid or rigid tabs and/or strut-like linking arms connected by flexure joints at one or more locations along the edges of the positive and negative lenses in order to secure the lenses independently within the eye's ciliary sulcus or lens capsule.

6. The eye intra ocular lens system of claim 5 wherein there is only said positive lens means has a hinge mechanism which controls the movement of said positive lens in response to the movement of the ciliary muscle of the eye acting on the hinge mechanism during the accommodation response, and said positive and negative lens are linked mechanically.

7. The eye intra ocular lens system of claim 1 wherein said positive lens means has a frame and at least two pairs of haptics that controls the movement of said positive lens in response to the movement of the ciliary muscle of the eye acting on the haptics during the accommodation response, and with one pair of haptics having one end hinged to the lower half of said frame and the second end connected to an upper portion of the ciliary muscle, and a second pair of haptics hinged to an upper half of said frame and to a lower potion of the ciliary muscle.

8. The eye intra ocular lens system of claim 7, wherein the one pair of haptics extend substantially parallel and the second pair of haptics extend substantially parallel.

9. An intra ocular lens comprising:
a negative lens and a positive lens that are axially separated and said intra ocular lens is formed inside the eye as part of an implantation of the negative and positive lenses in an eye or outside of the eye by connecting the negative and positive lenses prior to implantation into the eye, and
said positive lens has positive lens means to move said positive lens relative to said negative lens in response to movement of the ciliary muscle of the eye during accommodation response of the eye and said movements during the accommodation response are along the optical axis of the eye and are controlled in order to improve the image on the retina of objects viewed by the eye over a wide range of distances.

10. The intra ocular lens of claim 9 wherein said positive lens means has a frame and at least two pairs of haptics that controls the movement of said positive lens in response to the movement of the ciliary muscle of the eye acting on the haptics during the accommodation response, and with one pair of haptics having one end hinged to the lower half of said frame and the second end connected to an upper portion of the ciliary muscle, and a second pair of haptics hinged to an upper half of said frame and to a lower potion of the ciliary muscle.

11. The intra ocular lens of claim 10 wherein the one pair of haptics extend substantially parallel and the second pair of haptics extend substantially parallel.

12. An intra ocular lens comprising:
an optic lens,
at least two pairs of haptics that controls the movement of said optic lens along the optical axis of the eye in response to the movement of the ciliary muscle of the eye acting on the haptics during the accommodation response, one pair of hautics having one end hinged to the lower half of said optic lens and the second end connected to an upper portion of the ciliary muscle, and a second pair of haptics hinged to an upper half of said optic lens and to a lower potion of the ciliary muscle.

13. The intra ocular lens of claim 11 wherein the one pair of haptics extend substantially parallel and the second pair of haptics extend substantially parallel.

14. The intra ocular lens of claim 12 wherein the one pair and second pair of haptics are attached to a frame surrounding the optic lens with the one pair one pair of haptics having one end hinged to the lower potion said frame and the second end connected to an upper portion of the ciliary muscle, and the second pair of haptics hinged to an upper half of said frame and to a lower potion of the ciliary muscle.

15. A method of improving vision for an eye which has been diagnosed as being approved for intra ocular lens implants comprising implanting a negative lens with, before or after implanting a positive lens, and implanting said positive lens such that the positive lens will move relative to negative lens in response to movement of the ciliary muscle of the eye during accommodation response of the eye.

16. The method as claimed in claim 15 wherein said negative lens and positive lens form an intra ocular lens, providing said positive lens with positive lens means to move said positive lens relative to said negative lens in response to movement of the ciliary muscle of the eye during accommodation response of the eye and said movements during the accommodation response are along the optical axis of the eye and are controlled in order to improve the image on the retina of objects viewed by the eye over a wide range of distances.

17. The method of claim 16 comprising implanting the positive lens in either the eye's ciliary sulcus or lens capsule and implanting the negative lens in either the eye's ciliary sulcus or lens capsule.

18. The method as claimed in any one of claims 17 wherein the positive and negative lenses can have any of the following types of surface shapes: spherical, astigmatic toric, aspherical with or without axial symmetry, and multi-zoned surfaces as those found on Fresnel lenses, diffractive surfaces, and one surface diffractive and the other surface diffractive.

19. The method of claim 18 wherein the intra ocular lens is a single optic lens having at least two pairs of haptics that controls the movement of said optic lens along the optical axis of the eye in response to the movement of the ciliary muscle of the eye acting on the haptics during the accommodation response, one pair of haptics having one end hinged to the lower half of said optic lens and the second end connected to an upper portion of the ciliary muscle, and a second pair of haptics hinged to an upper half of said optic lens and to a lower potion of the ciliary muscle.

20. The method claim 19 wherein the one pair and second pair of haptics are attached to a frame surrounding the optic lens with the one pair of haptics having one end hinged to the lower potion said frame and the second end connected to an upper portion of the ciliary muscle, and the second pair of haptics hinged to an upper half of said frame and to a lower potion of the ciliary muscle, the one pair of haptics extend substantially parallel, and the second pair of haptics extend substantially parallel.
